(19) United States
(12) Patent Application Publication
(10) Pub. No.: US 2023/0380968 A1
(43) Pub. Date: Nov. 30, 2023

(54) TRANSCATHETERAL DEPLOYMENT OF A MEDICAL DEVICE WITHOUT USE OF A NOSE CONE

(71) Applicant: Tel Hashomer Med. Res., Infra. & Serv. LTD., Ramat-Gan (IL)

(72) Inventor: Boris Orlov, Rishon Letzion (IL)

(21) Appl. No.: 18/248,220

(22) PCT Filed: Oct. 24, 2021
(86) PCT No.: PCT/IB2021/059798

§ 371 Date: Apr. 6, 2023

Related U.S. Application Data
(60) Provisional application No. 63/106,000, filed on Oct. 27, 2020.

(51) Int. Cl. A61F 2/24

(52) U.S. Cl. CPC A61F 2/2439; A61F 2/2436; A61F 2/2418; A61F 2002/9665

(57) ABSTRACT

Apparatus and methods are described including a medical device (22, 90), and a delivery catheter (24) that is configured deliver the medical device (22, 90) to a portion of subjects body. An overtube (40) is configured to maintain a proximal portion of the medical device (22, 90) in a radially-constrained configuration, during delivery of the medical device (22, 90) to the portion of the subjects body. A string-rod constraining mechanism (41) includes a rod (44) disposed at least partially within the overtube (40), and at least one string (42). The string-rod mechanism (41) maintains a distal portion of the medical device (22, 90) in a radially-constrained configuration, when the overtube (40) is not covering the distal portion of the medical device (22, 90), by the at least one string (42) extending from the distal portion of the medical device to the rod (44). Other applications are also described.

### CROSS-REFERENCES TO RELATED APPLICATIONS

The present application claims priority from U.S. Provisional Patent Application 63/106,000 to Orlov, filed Oct. 27, 2020, entitled “Transcatheteral deployment of a medical device without use of a nose cone,” which is incorporated herein by reference.

### FIELD OF EMBODIMENTS OF THE INVENTION

The present invention relates to medical apparatus and methods, and specifically to apparatus and methods for deploying an implantable medical device.

### BACKGROUND

There are many implantable medical devices that are delivered percutaneously, using a catheter. Typically, such devices are delivered in a radially-constrained (also known as a “crimped”) configuration, and become deployed by being radially expanded. Some such devices are configured to self-expand, while other devices are configured to be radially expanded in an active manner. In some cases, it is desirable for a proximal portion of a transcatheterally-delivered medical device to assume a non-radially constrained configuration while a distal portion is still maintained in a radially constrained configuration. Typically, in such cases a nose cone is held over the distal portion of the device to maintain the distal portion in a radially-constrained state, while the proximal portion is released from the catheter.

### SUMMARY OF EMBODIMENTS

In accordance with some applications of the present invention, a transcatheterally-delivered medical device has a proximal portion that is configured to assume a non-radially constrained configuration, while a distal portion is still to be maintained in a radially constrained configuration. For example, the medical device may be a stent. Alternatively, the medical device may be a prosthetic mitral valve that includes a valve frame having a valve-frame body that defines a ventricular portion (which upon deployment is configured to be disposed within the subject's left ventricle), and an atrial portion (which upon deployment is configured to be disposed within the subject's left atrium). The prosthetic mitral valve typically includes a plurality of leaflets (e.g., two leaflets, or three leaflets, as shown), which are sutured or otherwise coupled to the valve-frame body. For some applications, in a non-constrained configuration of the prosthetic mitral valve frame, a plurality of chord-recruiting arms (e.g., more than two and/or fewer than fifteen arms) extend radially from a portion of valve-frame body that is configured to be placed within the subject's ventricle.

Typically, the prosthetic mitral valve and the prosthetic mitral valve frame are delivered to the subject's native mitral valve, using a delivery catheter, and the delivery catheter is configured to maintain the prosthetic mitral valve and the prosthetic mitral valve frame in radially-constrained configurations (i.e., “crimped” configurations) during the delivery. In accordance with respective applications, the prosthetic mitral valve and the prosthetic mitral valve frame are delivered transseptally (i.e., via the vena cava, the right atrium, and the interatrial septum), transapically (i.e., via the apex of the left ventricle), and/or via a different delivery path.

Typically, during delivery of the prosthetic mitral valve frame to the subject's native mitral valve, the proximal end of the valve-frame body is maintained in its radially constrained configuration by an overtube of the delivery catheter that is disposed over the proximal end of valve-frame body and which prevents the proximal end of valve-frame body from radially expanding. Further typically, during the delivery of the prosthetic mitral valve frame to the native mitral valve, the distal end of the of valve-frame body is maintained in a radially-constrained configuration by a string-rod constraining mechanism. The string-rod constraining mechanism typically includes at least one string that extends from the distal end of the valve-frame body to a rod that is disposed at least partially within the overtube of the delivery catheter (e.g., a rod disposed along a longitudinal axis of the delivery catheter, as shown). For some applications, the at least one string comprises a plurality of loops that extend from the distal end of the valve-frame body, and that radially constrain the distal end of the valve-frame body by looping around the rod.

Typically, the string-rod constraining mechanism maintains the distal end of the valve-frame body in its radially-constrained configuration, even after the chord-recruiting arms are allowed to assume non-radially-constrained configurations by expanding radially (by virtue of the overtube being retracted from over the chord-recruiting arms), as described in further detail hereinbelow. An alternative method for maintaining the distal end of the valve-frame body in its radially-constrained configuration even after the chord-recruiting arms are allowed to expand radially would be to use a nose cone to cover the distal end of the valve-frame body. However, it would then be necessary to retract the nose cone through the prosthetic mitral valve, which may risk damaging the prosthetic mitral valve and/or dislodging the prosthetic mitral valve frame. It is noted that the retraction of the nose cone through the mitral valve is from the left ventricle to the left atrium, which is the direction in which the prosthetic mitral valve leaflets are configured to block blood flow, making the prosthetic mitral valve leaflets particularly susceptible to damage. Using the string-rod constraining mechanism to maintain the distal end of the valve-frame body in its radially-constrained configuration, rather than using a nose cone, typically reduces a risk of damaging the prosthetic mitral valve and/or dislodging the prosthetic mitral valve frame in this manner. This is because the rod typically has a relatively small diameter, and the strings typically remain coupled to a portion of the prosthetic mitral valve frame and are not removed from the subject's heart. Thus, the rod can be retracted through the prosthetic mitral valve without causing any damage to, or dislodging, the prosthetic mitral valve, and the strings do not need to be retracted via the prosthetic mitral valve.

Although some applications of the present invention are described as being utilized in conjunction with a particular type of prosthetic mitral valve and prosthetic mitral valve frame, the scope of the present invention includes using generally similar apparatus and techniques with any prosthetic mitral valve and prosthetic mitral valve frame. Similarly, although some applications of the present invention are described as being utilized in conjunction with a prosthetic mitral valve and a prosthetic mitral valve frame, the scope of the present invention includes using generally similar apparatus and techniques with any prosthetic atrioventricular valve and prosthetic atrioventricular valve. Thus, the scope of the present invention includes using generally similar apparatus and techniques with a prosthetic tricuspid valve and prosthetic tricuspid valve frame having a generally similar configuration to the prosthetic mitral valve and the prosthetic mitral valve frame described herein, mutatis mutandis. Similarly, although some applications of the present invention are described as being utilized in conjunction with a prosthetic valve and a prosthetic valve frame, the scope of the present invention includes using generally similar apparatus and techniques with any transcatheterally-delivered medical device having a proximal portion that is configured to assume a non-radially constrained configuration while a distal portion is still to be maintained in a radially constrained configuration (e.g., a stent, as described herein), mutatis mutandis. Typically, during the delivery of the device by the delivery catheter, the proximal portion is maintained in a radially-constrained configurations by the overtube of the delivery catheter, and the distal portion of the device is maintained in a radially-constrained configuration using a string-rod constraining mechanism, in a generally similar manner to that described hereinabove. For some such applications, a plurality of loops extend from the distal portion of the device, and radially constrain the distal portion of the device by looping the rod. The proximal portion is allowed to assume its non-radially-constrained configuration by retracting the overtube from over the proximal portion, while the distal portion of the device is maintained in its radially-constrained configuration using the string-rod constraining mechanism. Subsequently, the distal portion of the device is allowed to assume its non-radially-constrained configuration by retracting the rod, to thereby release the at least one string (e.g., the loops) of the string-rod constraining mechanism.

There is therefore provided, in accordance with some applications of the present invention, apparatus including:
a medical device; and
a delivery catheter that is configured deliver the medical device to a portion of a body of a subject, the delivery catheter comprising:
   an overtube configured, during delivery of the medical device to the portion of the subject's body, to maintain a proximal portion of the medical device in a radially-constrained configuration by covering the proximal portion of the medical device; and
   a string-rod constraining mechanism comprising:
      a rod disposed at least partially within the overtube; and
      at least one string,
      the string-rod mechanism being configured to maintain a distal portion of the medical device in a radially-constrained configuration, when the overtube is not covering the distal portion of the medical device, by the at least one string extending from the distal portion of the medical device to the rod.

In some applications, the delivery catheter is configured to release the distal portion of the medical device from being maintained in the radially-constrained configuration by the rod being retracted such as to release the at least one string.

In some applications, the at least one string includes a plurality of loops extending from the distal portion of the medical device and that are configured to maintain the distal portion of the medical device in the radially-constrained configuration by looping around the rod.

In some applications, the delivery catheter is configured to maintain the distal portion of the medical device in the radially-constrained configuration when the overtube is not covering the distal portion of the medical device, without using a nose cone to maintain the distal portion of the medical device in the radially-constrained configuration.

In some applications, the medical device includes a prosthetic tricuspid valve, the prosthetic tricuspid valve including:
a valve-frame body that defines a ventricular portion that is configured to de disposed within a right ventricle of the subject, and atrial portion that is configured to be disposed inside a right atrium of the subject;
a plurality of leaflets that are coupled to the valve frame body; and
a plurality of chord-recruiting arms that are configured to extend radially from the ventricular portion of the valve frame body.

In some applications, the medical device includes a prosthetic mitral valve, the prosthetic mitral valve including:
a valve-frame body that defines a ventricular portion that is configured to de disposed within a left ventricle of the subject, and atrial portion that is configured to be disposed inside a left atrium of the subject;
a plurality of leaflets that are coupled to the valve frame body; and
a plurality of chord-recruiting arms that are configured to extend radially from the ventricular portion of the valve frame body.

In some applications:
the prosthetic mitral valve is configured to be delivered to a native mitral valve of the subject from above the native mitral valve,
during the delivery of the prosthetic mitral valve to the subject's native mitral valve, the ventricular portion of the valve frame body is configured to be disposed distally with respect to the atrial portion of the valve-frame body, within the delivery catheter, and
the at least one string is configured to extend from a distal end of the ventricular portion of the valve-frame body to the rod.

In some applications, the at least one string includes a plurality of loops extending from the distal end of the ventricular portion of the valve-frame body and that are configured to maintain the ventricular portion of the valve frame body in a radially-constrained configuration by looping around the rod.

In some applications:
the prosthetic mitral valve is configured to be delivered to a native mitral valve of the subject from below the native mitral valve,
during the delivery of the prosthetic mitral valve to the subject's native mitral valve, the atrial portion of the valve frame body is configured to be disposed distally with respect to the ventricular portion of the valve-frame body, within the delivery catheter, and
the at least one string is configured to extend from a distal end of the atrial portion of the valve-frame body to the rod.

In some applications, the at least one string includes a plurality of loops extending from the distal end of the atrial portion of the valve-frame body, and that are configured to maintain the atrial portion of the valve frame body in a radially-constrained configuration by looping around the rod.

There is further provided, in accordance with some applications of the present invention, a method for use with a medical device including:
delivering the medical device to a portion of a body of a subject using a delivery catheter, while (a) a proximal portion of the medical device is maintained in a radially-constrained configuration by an overtube of the delivery catheter covering the proximal portion of the medical device, and (b) a distal portion of the medical device is maintained in a radially-constrained configuration, by a string-rod constraining mechanism that comprises at least one string that extends from the distal portion of the medical device to a rod that is disposed at least partially within the overtube; and
when a distal end of the delivery catheter is disposed at the portion of the subject's body:
   allowing the proximal portion of the medical device to assume a non-radially-constrained configuration, by retracting the overtube from over the proximal portion of the medical device, while maintaining the distal portion of the medical device in its radially-constrained configuration, by the at least one string extending from the distal portion of the medical device to the rod; and
   subsequently, allowing the distal portion of the medical device to assume a non-radially-constrained configuration, by retracting the rod, to thereby release the at least one string.

In some applications, the at least one string includes a plurality of loops extending from the distal portion of the medical device, and delivering the medical device to the portion of the subject's body using the delivery catheter includes delivering the medical device to the portion of the subject's body using the delivery catheter, while the distal portion of the medical device is maintained in the radially-constrained configuration by the plurality of loops looping around the rod.

In some applications, delivering the medical device to the portion of the subject's body, while the distal portion of the medical device is maintained in the radially-constrained configuration includes delivering the medical device to the portion of the subject's body, without using a nose cone to maintain the distal portion of the medical device in the radially-constrained configuration.

In some applications, delivering the medical device to the portion of the subject's body using the delivery catheter includes delivering a prosthetic tricuspid valve to the portion of the subject's body using the delivery catheter, the prosthetic tricuspid valve including:
a valve-frame body that defines a ventricular portion that is configured to be disposed within a right ventricle of the subject, and atrial portion that is configured to be disposed inside a right atrium of the subject;
a plurality of leaflets that are coupled to the valve frame body; and
a plurality of chord-recruiting arms that are configured to extend radially from the ventricular portion of the valve frame body.

In some applications, delivering the medical device to the portion of the subject's body using the delivery catheter includes delivering a prosthetic mitral valve to the portion of the subject's body using the delivery catheter, the prosthetic mitral valve including:
a valve-frame body that defines a ventricular portion that is configured to be disposed within a left ventricle of the subject, and atrial portion that is configured to be disposed inside a left atrium of the subject;
a plurality of leaflets that are coupled to the valve frame body; and
a plurality of chord-recruiting arms that are configured to extend radially from the ventricular portion of the valve frame body.

In some applications, delivering the prosthetic mitral valve to the portion of the subject's body using the delivery catheter includes delivering the prosthetic mitral valve to a native mitral valve of the subject, from above the subject's native mitral valve, with the ventricular portion of the valve frame body disposed distally with respect to the atrial portion of the valve-frame body within the delivery catheter, and with the at least one string extending from a distal end of the ventricular portion of the valve-frame body to the rod.

In some applications, the at least one string of the string-rod constraining mechanism includes a plurality of loops extending from the distal end of the ventricular portion of the valve-frame body, and delivering the prosthetic mitral valve to the subject's native mitral valve includes delivering the prosthetic mitral valve to the subject's native mitral valve, while the distal end of the ventricular portion of the valve-frame body is maintained in the radially-constrained configuration by the plurality of loops looping around the rod.

In some applications, delivering the prosthetic mitral valve to the portion of the subject's body using the delivery catheter includes delivering the prosthetic mitral valve to a native mitral valve of the subject, from below the subject's native mitral valve, with the atrial portion of the valve frame body disposed distally with respect to the ventricular portion of the valve-frame body within the delivery catheter, and with the at least one string extending from a distal end of the atrial portion of the valve-frame body to the rod.

In some applications, the at least one string of the string-rod constraining mechanism includes a plurality of loops extending from the distal end of the atrial portion of the valve-frame body, and delivering the prosthetic mitral valve to the subject's native mitral valve includes delivering the prosthetic mitral valve to the subject's native mitral valve, while the distal end of the atrial portion of the valve-frame body is maintained in the radially-constrained configuration by the plurality of loops looping around the rod.

The present invention will be more fully understood from the following detailed description of applications thereof, taken together with the drawings, in which:

### BRIEF DESCRIPTION OF THE DRAWINGS

FIG. 1 is a schematic illustration of a prosthetic mitral valve and a prosthetic mitral valve frame being placed inside a delivery catheter with a ventricular portion of the prosthetic mitral valve frame disposed toward a distal end of the delivery catheter, in accordance with some applications of the present invention;

FIGS. 2A, 2B, 2C, 2D, 2E, and 2F are schematic illustrations of respective steps of the deployment of the prosthetic mitral valve and the prosthetic mitral valve frame at a subject's native mitral valve, via a transseptal delivery approach, in accordance with some applications of the present invention;

FIG. 3 is a schematic illustration of a prosthetic mitral valve and a prosthetic mitral valve frame being placed inside a delivery catheter with an atrial portion of the prosthetic mitral valve frame disposed toward a distal end of the delivery catheter, in accordance with some applications of the present invention;

FIGS. 4A, 4B, 4C, 4D, 4E, and 4F are schematic illustrations of respective steps of the deployment of the prosthetic mitral valve and the prosthetic mitral valve frame at a subject's native mitral valve, via a transapical delivery approach, in accordance with some applications of the present invention;

FIG. 5 is a schematic illustration of the distal end of the of a valve-frame body of a prosthetic mitral valve frame being maintained in its radially-constrained configuration using a nose cone to cover the distal end of the valve-frame body;

FIGS. 6A and 6B are schematic illustrations of a segmented nose cone, which is used to penetrate a subject's interatrial septum, in accordance with some applications of the present invention; and

FIGS. 7A, 7B, and 7C are schematic illustrations of respective steps of a stent being deployed within a blood vessel, in accordance with some applications of the present invention.

### DETAILED DESCRIPTION OF EMBODIMENTS

Reference is now made to FIG. 1, which is a schematic illustration of a prosthetic mitral valve 20 and a prosthetic mitral valve frame 22 being placed inside a delivery catheter 24 with a ventricular portion 26 of the prosthetic mitral valve frame disposed toward a distal end 28 of the delivery catheter, in accordance with some applications of the present invention. Typically, the prosthetic mitral valve frame includes a valve-frame body 30 that defines ventricular portion 26 (which upon deployment is configured to be disposed within the subject's left ventricle), and an atrial portion 32 (which upon deployment is configured to be disposed within the subject's left atrium). Prosthetic mitral valve 20 typically includes a plurality of leaflets 34 (e.g., two leaflets, or three leaflets, as shown), which are sutured or otherwise coupled to the valve-frame body.

Typically, valve frame 22 is made of a shape-memory material (e.g., a shape-memory alloy, such as nitinol and/or copper-aluminum-nickel), which is covered on one or both sides with a covering material 36, e.g., a fabric and/or a polymer (such as expanded polytetrafluoroethylene (ePTFE), or woven, knitted and/or braided polyester). Typically, the shape-memory material of valve frame is shaped into a stent-like structure that comprises struts and/or cells of the shape-memory material. The covering material is typically coupled to the shape-memory material via stitches.

For some applications, in a non-constrained configuration of prosthetic mitral valve frame 22, a plurality of chord-recruiting arms 38 (e.g., more than two and/or fewer than fifteen arms) extend radially from a portion of valve-frame body 30 that is configured to be placed within the subject's ventricle. For example, four chord-recruiting arms or six chord-recruiting arms may extend from the valve-frame body. Typically, the arms are configured to extend radially from the valve-frame body, in addition to extending axially from a ventricular end of the valve-frame body (i.e., the end of the valve-frame body that is configured to be placed within the ventricle) toward an atrial end of the valve-frame body (i.e., the end of the valve-frame body that is configured to be placed within the atrium). Further typically, the arms curve around outside of the valve-frame body in a given circumferential direction of curvature.

Typically, prosthetic mitral valve 20 and prosthetic mitral valve frame 22 are delivered to the native mitral valve, using delivery catheter 24, and the delivery catheter is configured to maintain prosthetic mitral valve 20 and prosthetic mitral valve frame 22 in radially-constrained configurations (i.e., “crimped” configurations) during the delivery. In accordance with respective applications, the prosthetic mitral valve 20 and prosthetic mitral valve frame 22 are delivered transseptally (i.e., via the vena cava, the right atrium, and the interatrial septum), transapically (i.e., via the apex of the left ventricle), and/or via a different delivery path.

The right side of FIG. 1 shows the prosthetic mitral valve 20 and prosthetic mitral valve frame 22 disposed within delivery catheter 24 in a configuration that is suitable for transseptal delivery. In this configuration, ventricular portion 26 of the prosthetic mitral valve frame is disposed toward distal end 28 of the delivery catheter. Typically, the proximal end of valve-frame body 30 is maintained in its radially constrained configuration by an overtube 40 of the delivery catheter that is disposed over the proximal end of valve-frame body 30 and which prevents the proximal end of valve-frame body 30 from radially expanding.

Further typically, during the delivery of the prosthetic mitral valve frame to the native mitral valve, the distal end of the of valve-frame body 30 (which in the configuration shown in FIG. 1 is at the ventricular end of the prosthetic mitral valve frame) is maintained in its radially-constrained configuration by a string-rod constraining mechanism 41. String-rod constraining mechanism 41 typically includes at least one string 42, e.g., a plurality of strings, that extend from the distal end of the valve-frame body to a rod 44 that is disposed at least partially within the overtube of the delivery catheter (e.g., a rod disposed along a longitudinal axis of the delivery catheter, as shown). For some applications, the at least one string includes a plurality of loops 46 that extend from the distal end of the valve-frame body, and that radially constrain the distal end of the valve-frame body by looping around rod 44. Typically, string-rod constraining mechanism 41 maintains the distal end of the valve-frame body in its radially-constrained configuration, even after chord-recruiting arms 38 are allowed to assume non-radially-constrained configurations by expanding radially (by virtue of overtube 40 being retracted from over the chord-recruiting arms), as described in further detail hereinbelow. An alternative method for maintaining the distal end of the valve-frame body in its radially-constrained configuration even after chord-recruiting arms 38 are allowed to expand radially would be to use a nose cone 74 to cover the distal end of the valve-frame body, e.g., as shown in FIG. 5. However, it would then be necessary to retract the nose cone through the prosthetic mitral valve, which may risk damaging the prosthetic mitral valve and/or dislodging the prosthetic mitral valve frame. It is noted that the retraction of the nose cone through the mitral valve is from the left ventricle to the left atrium, which is the direction in which the prosthetic mitral valve leaflets are configured to block blood flow, making the prosthetic mitral valve leaflets particularly susceptible to damage. Using string-rod constraining mechanism 41 to maintain the distal end of the valve-frame body in its radially-constrained configuration, rather than using a nose cone, typically reduces a risk of damaging the prosthetic mitral valve and/or dislodging the prosthetic mitral valve frame in this manner. This is because the rod typically has a relatively small diameter, and the strings (e.g., loops 46 of string) typically remain coupled to a portion of the prosthetic mitral valve frame and are not removed from the subject's heart. The diameter of the rod is typically less than 9 French (3 mm), e.g., less than 6 French (2 mm). Thus, the rod can be retracted through the prosthetic mitral valve without causing any damage to or dislodging the prosthetic mitral valve, and the strings do not need to be retracted via the prosthetic mitral valve.

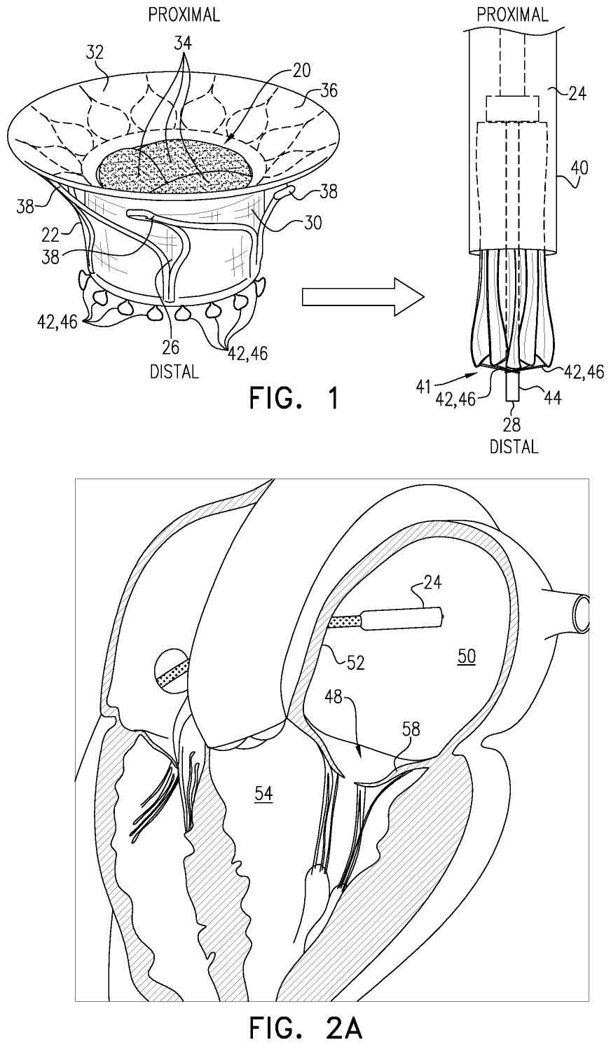

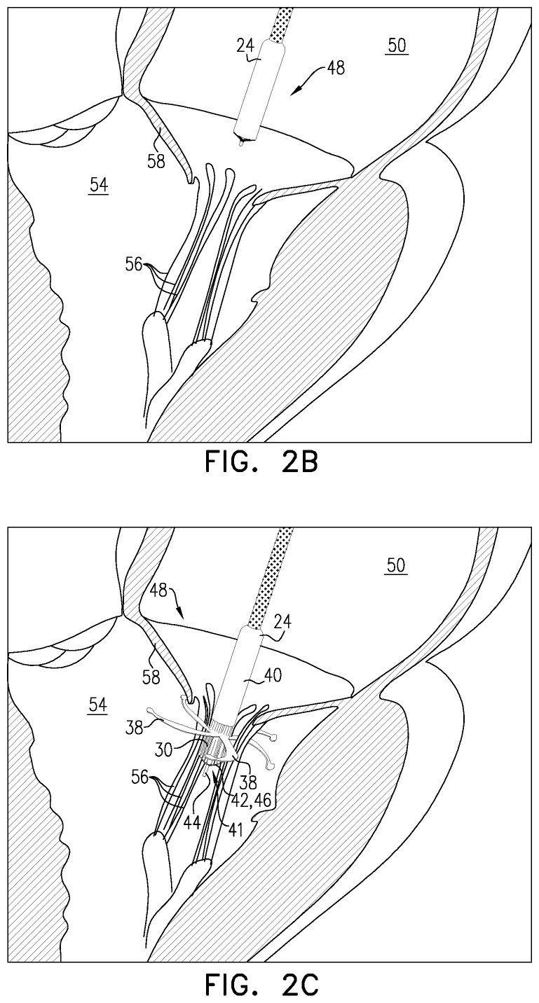

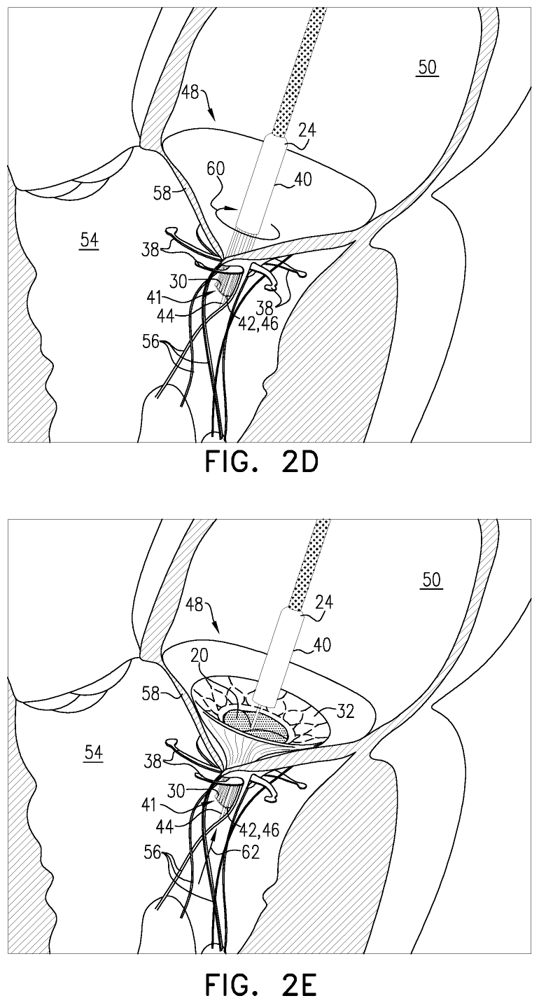

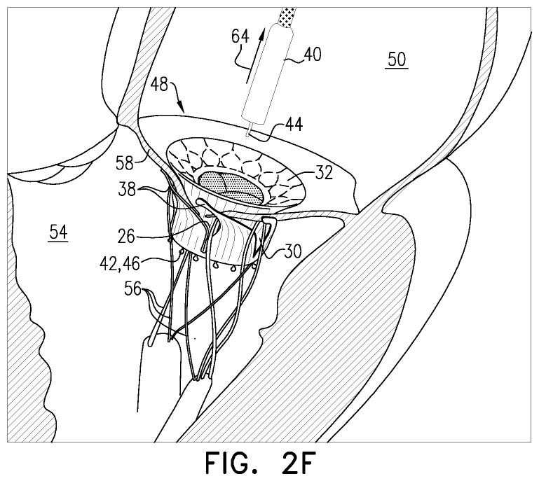

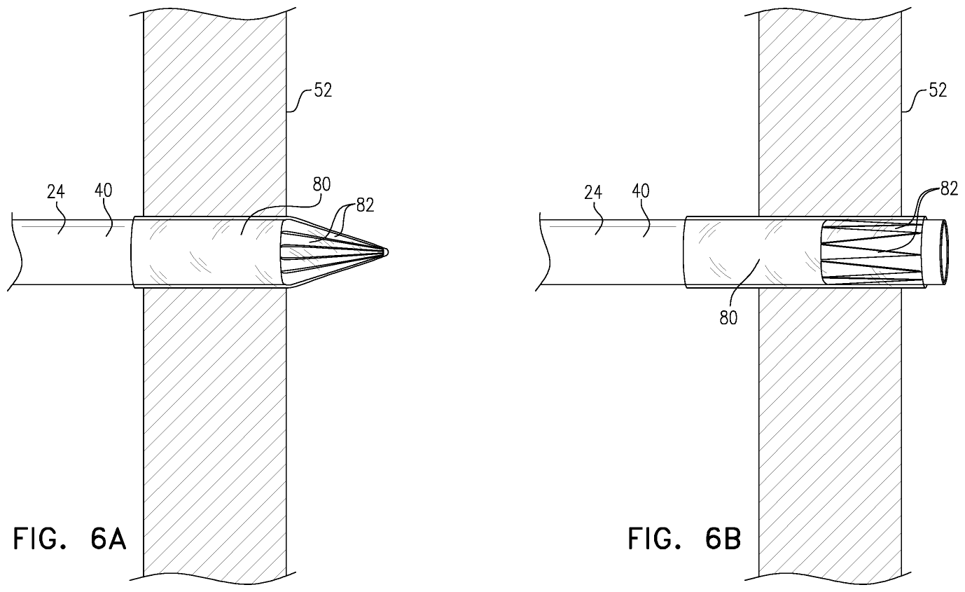

Reference is now made to FIGS. 2A, 2B, 2C, 2D, 2E, and 2F, which are schematic illustrations of respective steps of the deployment of prosthetic mitral valve 20 and prosthetic mitral valve frame 22 at a subject's native mitral valve 48, via a transseptal delivery approach, in accordance with some applications of the present invention. As shown in FIG. 2A, the distal end of delivery catheter 24 is typically advanced into the subject's left atrium 50, via the interatrial septum 52. For some applications, in order for the distal end of the delivery catheter to penetrate the interatrial septum, a nose cone is disposed over the distal end of the delivery catheter for this stage. Subsequent to the interatrial septum having been penetrated and before advancing the distal end of the delivery catheter into the left ventricle, the nose cone is retracted proximally from the left atrium. Alternatively or additionally, in order for the distal end of the delivery catheter to penetrate the interatrial septum, an outer shaft (not shown) is placed through the interatrial septum (e.g., using techniques that are known in the art) such that the distal end of the outer shaft is disposed in the left atrium, and the distal end of the delivery catheter is advanced to the left atrium via the outer shaft. Further alternatively or additionally, a segmented nose cone 80 is disposed over the distal end of the delivery catheter and is used to penetrate the interatrial septum, as shown in FIGS. 6A-B and described in further detail hereinbelow. Subsequent to penetration of the septum, the segmented nose cone is retracted proximally, such that segments 82 of the segmented nose cone become separated from each other.

Subsequent to the above-described step, the distal end of the delivery catheter is advanced toward the native mitral valve, as shown in FIG. 2B, and then is advanced through leaflets 58 of the native mitral valve and into left ventricle 54. For some applications (not shown), during the advancement of the distal end of the delivery catheter toward the native mitral valve and then through leaflets 58 of the native mitral valve and into left ventricle 54, the distal end of the delivery catheter and/or the distal end of rod 44 is covered with an elastic sheath (e.g., a condom or condom-like sheath), in order to prevent causing trauma to tissue of the subject's heart (e.g., the native mitral valve leaflets). Typically, for such applications, the elastic sheath is placed over the distal end of the delivery catheter and/or the distal end of rod 44 in a highly stretched state. Further typically, the elasticity of the sheath is such that, once the sheath is removed from the distal end of the delivery catheter and/or the distal end of rod 44, the sheath reduces in size substantially. Thus, the sheath may be readily retracted through the prosthetic mitral valve without causing any damage to or dislodgement of the prosthetic mitral valve.

When the distal end of the delivery catheter is disposed within the left ventricle, overtube of the delivery catheter is partially retracted, such that chord-recruiting arms 38 are allowed to assume non-radially-constrained configurations and to at least partially radially expand, as shown in FIG. 2C. Typically, at this stage, the distal end of valve-frame body 30 is maintained in its radially-constrained configuration by strings 42 (e.g., loops 46) and rod 44 of string-rod constraining mechanism 41, as shown in FIG. 2C. Typically, the chord-recruiting arms are shape set to extend radially from valve-frame body 30, upon being released from the overtube of delivery catheter 24.

As shown in FIG. 2D, subsequent to chord-recruiting arms 38 being deployed among chords 56 of the native mitral valve (and typically while the distal end of valve-frame body 30 is still maintained in its radially-constrained configuration by strings 42 and rod 44), at least a portion of valve frame 22 is rotated in the direction of arrow 60, such as to cause chord-recruiting arms 38 to (a) pull the native atrio-ventricular valve radially inward toward the valve frame, and (b) twist the native atrio-ventricular valve around the valve frame, by recruiting and deflecting at least a portion of the chords. Typically, chord-recruiting arms 38 are configured to curve in a given circumferential direction with respect to the longitudinal axis of the valve frame. For example, the arms may curve in a clockwise direction or in a counter-clockwise direction with respect to the longitudinal axis of the valve frame. Typically, subsequent to chord-recruiting arms 38 being deployed among chords 56 of the native mitral valve, the valve frame is rotated in the same circumferential direction as the direction of the circumferential curvature of the arms. In the example shown in FIG. 2D, the arms curve in the clockwise circumferential direction, and the valve frame is rotated in this direction.

As shown in FIG. 2E, subsequent to the rotation of the valve-frame, atrial portion 32 of the prosthetic mitral valve frame is typically allowed to assume its non-radially-constrained configuration and to radially expand, by further retracting overtube 40 of delivery catheter 24. Subsequently (or simultaneously therewith, or prior thereto), rod 44 is retracted (as indicated by arrow 62 in FIG. 2E), causing strings 42 (e.g., loops 46) to be released from the rod and allowing the distal end of valve-frame body 30 and ventricular portion 26 of the valve frame to assume their non-radially constrained configurations. Strings 42 (e.g., loops 46) typically remain coupled to valve-frame body 30, as shown. FIG. 2F shows both ventricular portion 26 and atrial portion 32 of valve-frame body 30 in their non-radially-constrained (i.e., radially expanded) configurations. Typically, by ventricular portion 26 and atrial portion 32 both assuming their non-radially-constrained configurations, the valve frame is configured to trap the native valve leaflets 58 in a partially closed and twisted configuration, to thereby at least partially seal a space between the native mitral valve and the prosthetic valve. For example, the ventricular portion may be configured to radially expand such as to trap the native valve leaflets between the ventricular portion and the chord-recruiting arms, and/or the atrial portion may be configured to radially expand such as to trap the native valve leaflets between the atrial portion and the chord-recruiting arms.

Subsequent to rod 44 being retracted so as to release strings 42, the rod is further retracted through the prosthetic mitral valve. Delivery catheter 24 is typically then retracted in its entirety from the subject's left atrium, as indicated by arrow 64 in FIG. 2F. As noted hereinabove, an alternative method for maintaining the distal end of the valve-frame body in its radially-constrained configuration even after chord-recruiting arms 38 are allowed to expand radially would be to use a nose cone 74 to cover the distal end of the valve-frame body (as shown in FIG. 5). However, it would then be necessary to retract the nose cone through the prosthetic mitral valve, which may risk damaging the prosthetic mitral valve and/or dislodging the prosthetic mitral valve frame. Using string-rod constraining mechanism 41 to maintain the distal end of the valve-frame body in its radially-constrained configuration, rather than using a nose cone, typically reduces a risk of damaging the prosthetic mitral valve and/or dislodging the prosthetic mitral valve frame in this manner. This is because the rod typically has a relatively small diameter, and the strings (e.g., loops 46) typically remain coupled to a portion of the prosthetic mitral valve frame and are not removed from the subject's heart. The diameter of the rod is typically less than 9 French (3 mm), e.g., less than 6 French (2 mm). Thus, the rod can be retracted through the prosthetic mitral valve without causing any damage to or dislodging the prosthetic mitral valve, and the strings do not need to be retracted via the prosthetic mitral valve.

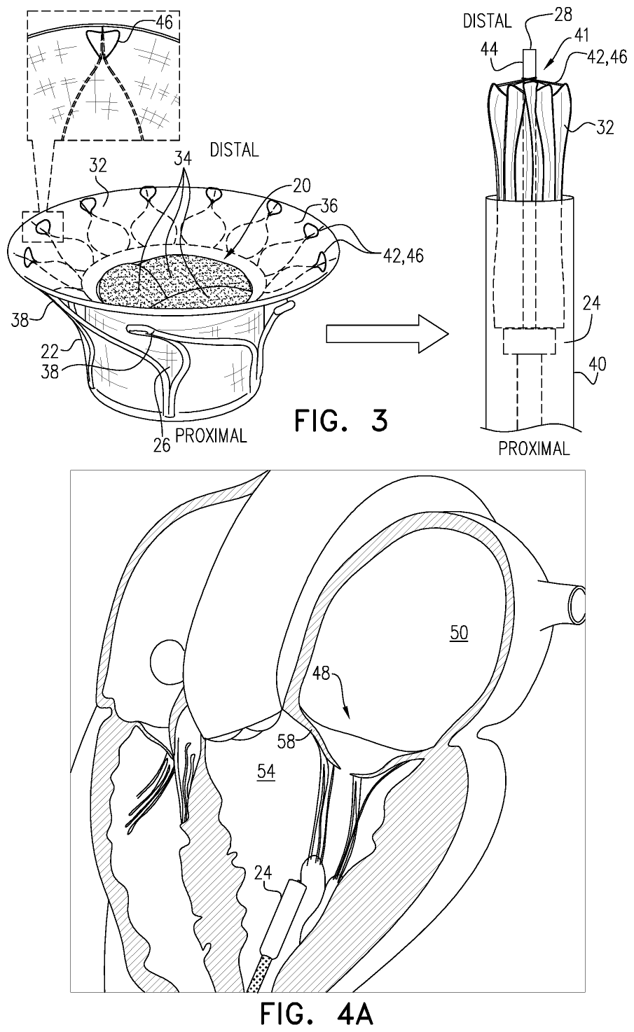

Reference is now made to FIG. 3, which is a schematic illustration of prosthetic mitral valve 20 and prosthetic mitral valve frame 22 being placed inside delivery catheter 24 with atrial portion 32 of the prosthetic mitral valve frame disposed toward distal end 28 of the delivery catheter, in accordance with some applications of the present invention. Typically, prosthetic mitral valve frame 22 is configured as shown in FIG. 3 for applications in which the delivery catheter is advanced toward the subject's native mitral valve transapically. In general, prosthetic mitral valve 20, prosthetic mitral valve frame 22, and delivery catheter 24 as shown in FIG. 3, are similar to these elements as described hereinabove, except for the differences described below.

As shown, when configured for transapical delivery, the end of atrial portion 32 is disposed toward distal end 28 of delivery catheter 24. In this configuration, string-rod constraining mechanism 41 is used to maintain the atrial portion in its radially-constrained configuration (in a generally similar manner to that described hereinabove with respect to ventricular portion 26), and overtube 40 is used to maintain chord-recruiting arms 38 and ventricular portion 26 in their radially-constrained configurations (in a generally similar manner to that described hereinabove with respect to chord-recruiting arms 38 and atrial portion 32). For some such applications, the at least one string 42 of the string-rod constraining mechanism includes a plurality of loops 46 that extend from the distal end of the valve-frame body (which in this case is at the end of atrial portion 32), and that radially constrain the distal end of the valve-frame body by looping around rod 44.

As described hereinabove, typically, strings 42 (e.g., loops 46) and rod 44 maintain the distal end of the valve-frame body in its radially-constrained configuration, even after chord-recruiting arms 38 are allowed to assume their non-radially-constrained configurations by expanding radially (by virtue of overtube 40 being retracted from over the chord-recruiting arms). An alternative method for maintaining the distal end of the valve-frame body in its radially-constrained configuration even after chord-recruiting arms 38 are allowed to expand radially would be to use a nose cone 74 to cover the distal end of the valve-frame body (as shown in FIG. 5). However, it would then be necessary to retract the nose cone through the prosthetic mitral valve, which may risk damaging the prosthetic mitral valve and/or dislodging the prosthetic mitral valve frame. Using string-rod constraining mechanism 41 to maintain the distal end of the valve-frame body in its radially-constrained configuration, rather than using a nose cone, typically reduces a risk of damaging the prosthetic mitral valve and/or dislodging the prosthetic mitral valve frame in this manner. This is because the rod typically has a relatively small diameter, and the strings (e.g., loops 46) typically remain coupled to a portion of the prosthetic mitral valve frame and are not removed from the subject's heart. The diameter of the rod is typically less than 9 French (3 mm), e.g., less than 6 French (2 mm). Thus, the rod can be retracted through the prosthetic mitral valve without causing any damage to or dislodging the prosthetic mitral valve, and the strings do not need to be retracted via the prosthetic mitral valve.

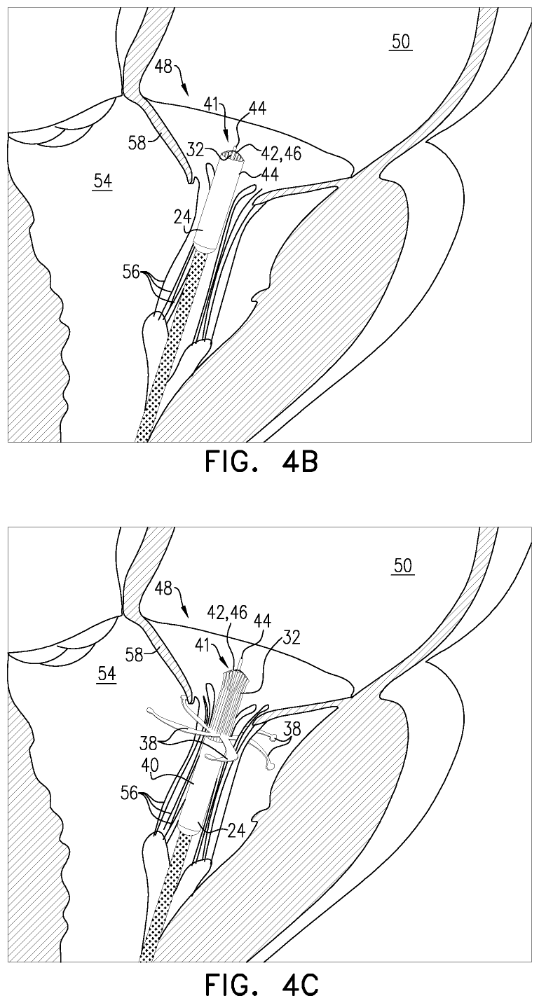

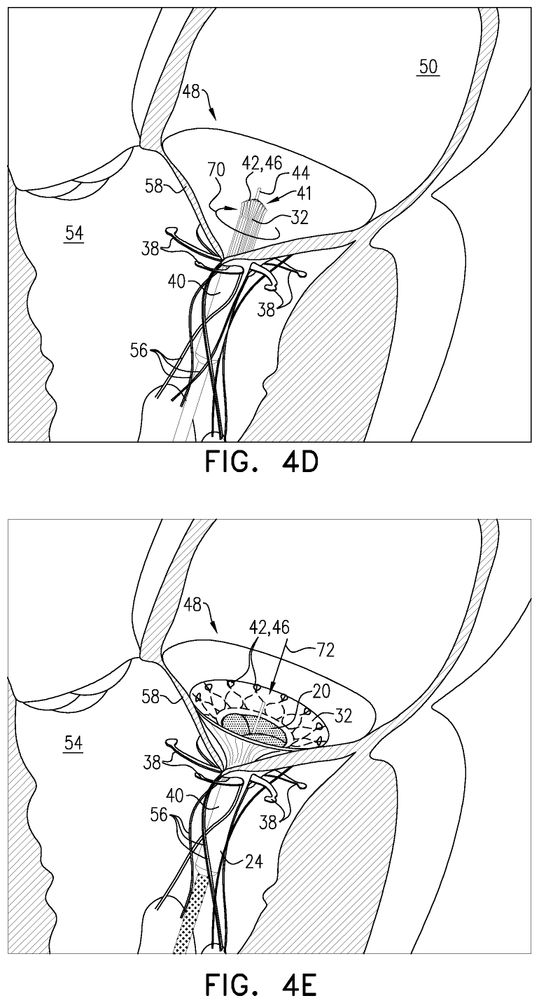

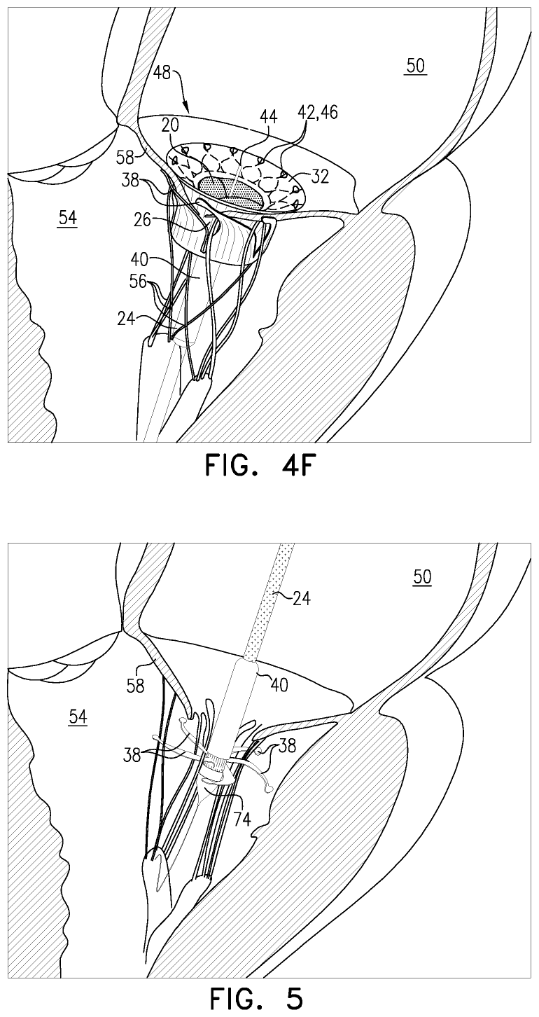

Reference is now made to FIGS. 4A, 4B, 4C, 4D, 4E, and 4F, which are schematic illustrations of respective steps of the deployment of prosthetic mitral valve 20 and prosthetic mitral valve frame 22 at a subject's native mitral valve 48, via a transapical delivery approach, in accordance with some applications of the present invention. As shown in FIG. 4A, the distal end of delivery catheter 24 is typically advanced into the subject's left ventricle, via the apex. For some applications, in order for the distal end of the delivery catheter to penetrate the apex, a technique is practiced that is similar to one of those described hereinabove for crossing the interatrial septum. For some applications, in order for the distal end of the delivery catheter to penetrate the apex, a nose cone is disposed over the distal end of the delivery catheter for this stage. Subsequent to the apex having been penetrated and before advancing the distal end of the delivery catheter into the left atrium, the nose cone is retracted proximally from the left ventricle. Alternatively or additionally, in order for the distal end of the delivery catheter to penetrate the apex, an outer shaft (not shown) is placed through the apex (e.g., using techniques that are known in the art) such that the distal end of the outer shaft is disposed in the left ventricle, and the distal end of the delivery catheter is advanced to the left ventricle via the outer shaft. Further alternatively or additionally, a segmented nose cone 80 is disposed over the distal end of the delivery catheter and is used to penetrate the apex, in a similar manner to that shown in FIGS. 6A-B with respect to the interatrial septum. Subsequent to penetration of the apex, the segmented nose cone is retracted proximally, such that segments 82 of the nose cone become separated from each other.

Subsequent to the above-described step, the distal end of the delivery catheter is advanced toward the native mitral valve, and continues to be advanced through leaflets 58 of the native mitral valve and into left atrium 50, as shown in FIG. 4B. For some applications (not shown), during the advancement of the distal end of the delivery catheter toward the native mitral valve and then through leaflets 58 of the native mitral valve and into left atrium 50, the distal end of the delivery catheter and/or the distal end of rod 44 is covered with an elastic sheath (e.g., a condom or condom-like sheath) in order to prevent causing trauma to tissue of the subject's heart (e.g., the native mitral valve leaflets). Typically, for such applications, the elastic sheath is placed over the distal end of the delivery catheter and/or the distal end of rod 44 in a highly stretched state. Further typically, the elasticity of the sheath is such that, once the sheath is removed from the distal end of the delivery catheter and/or the distal end of rod 44, the sheath reduces in size substantially. Thus, the sheath may be readily retracted through the prosthetic mitral valve without causing any damage to or dislodgement of the prosthetic mitral valve.

When the distal end of the delivery catheter is disposed within the left atrium, overtube 40 of the delivery catheter is partially retracted, such that chord-recruiting arms 38 are allowed to at least partially radially expand, as shown in FIG. 2C. Typically, at this stage, the distal end of valve-frame body 30 is maintained in its radially-constrained configuration by strings 42 (e.g., loops 46) and rod 44 of string-rod constraining mechanism 41, as shown in FIG. 4C. Typically, the chord-recruiting arms are shape set to extend radially from valve-frame body 30, upon being released from the overtube of delivery catheter 24.

As shown in FIG. 4D, subsequent to chord-recruiting arms 38 being deployed among chords of the native mitral valve (and typically while the distal end of valve-frame body 30 is still maintained in its radially-constrained configuration by strings 42 and rod 44), at least a portion of valve frame 22 is rotated in the direction of arrow 70, such as to cause chord-recruiting arms 38 to (a) pull the native atrio-ventricular valve radially inward toward the valve frame, and (b) twist the native atrio-ventricular valve around the valve frame, by recruiting and deflecting at least a portion of the chords. Typically, chord-recruiting arms 38 are configured to curve in a given circumferential direction with respect to the longitudinal axis of the valve frame. For example, the arms may curve in a clockwise direction or in a counter-clockwise direction with respect to the longitudinal axis of the valve frame. Typically, subsequent to chord-recruiting arms 38 being deployed among chords of the native mitral valve, the valve frame is rotated in the same circumferential direction as the direction of the circumferential curvature of the arms. In the example shown in FIG. 2D, the arms curve in the clockwise circumferential direction, and the valve frame is rotated in this direction.

As shown in FIG. 4E, subsequent to the rotation of the valve-frame, atrial portion 32 of the prosthetic mitral valve frame is typically allowed to assume its non-radially-constrained configuration and to radially expand, by retracting rod 44, to thereby cause strings 42 (e.g., loops 46) to be released (as indicated by arrow 72). Strings 42 (e.g., loops 46) typically remain coupled to valve-frame body 30, as shown. Subsequently (or simultaneously therewith, or prior thereto), ventricular portion 26 of the valve frame is allowed to assume its non-radially constrained configuration, by further retracting overtube 40 of delivery catheter 24. FIG. 4F shows both ventricular portion 26 and atrial portion 32 in their non-radially-constrained (i.e., radially expanded) configurations. Typically, by ventricular portion 26 and atrial portion 32 both assuming their non-radially-constrained configurations, the valve frame is configured to trap the native valve leaflets 58 in a partially closed and twisted configuration, to thereby at least partially seal a space between the native mitral valve and the prosthetic valve. For example, the ventricular portion may be configured to radially expand such as to trap the native valve leaflets between the ventricular portion and the chord-recruiting arms, and/or the atrial portion may be configured to radially expand such as to trap the native valve leaflets between the atrial portion and the chord-recruiting arms.

Subsequent to rod 44 being retracted so as to release strings 42 (e.g., loops 46), the rod is further retracted through the prosthetic mitral valve. Delivery catheter 24 is typically then retracted in its entirety from the subject's left ventricle. As noted hereinabove, an alternative method for maintaining the distal end of the valve-frame body in its radially-constrained configuration even after chord-recruiting arms 38 are allowed to expand radially would be to use a nose cone 74 to cover the distal end of the valve-frame body (e.g., as shown in FIG. 5). However, it would then be necessary to retract the nose cone through the prosthetic mitral valve, which may risk damaging the prosthetic mitral valve and/or dislodging the prosthetic mitral valve frame. Using string-rod constraining mechanism 41 to maintain the distal end of the valve-frame body in its radially-constrained configuration, rather than using a nose cone, typically reduces a risk of damaging the prosthetic mitral valve and/or dislodging the prosthetic mitral valve frame in this manner. This is because the rod typically has a relatively small diameter, and the strings (e.g., loops 46) typically remain coupled to a portion of the prosthetic mitral valve frame and are not removed from the subject's heart. The diameter of the rod is typically less than 9 French (3 mm), e.g., less than 6 French (2 mm). Thus, the rod can be retracted through the prosthetic mitral valve without causing any damage to or dislodging the prosthetic mitral valve, and the strings do not need to be retracted via the prosthetic mitral valve.

Reference is now made to FIG. 5, which is a schematic illustration of the distal end of the of valve-frame body 30 of prosthetic mitral valve frame 22 being maintained in its radially-constrained configuration using a nose cone 74 to cover the distal end of the valve-frame body. As described hereinabove, using nose cone 74 is an alternative method for maintaining the distal end of the valve-frame body in its radially-constrained configuration. However, once the distal end of the valve-frame body has been allowed to expand by advancing the nose cone, it is then necessary to retract the nose cone proximally through the prosthetic mitral valve. Due to the size and rigidity of the nose cone, this risks damaging the prosthetic mitral valve and/or dislodging the prosthetic mitral valve frame. In procedures such as that shown in FIG. 5, in which the nose cone would be retracted from the left ventricle into the left atrium, the prosthetic mitral valve leaflets are particularly susceptible to damage. This is because the nose cone the needs to be retracted through the prosthetic mitral valve leaflets in the direction in which the leaflets are configured to block blood flow.

Reference is now made to FIGS. 6A-B, which are schematic illustrations of segmented nose cone 80, which is used to penetrate interatrial septum 52, in accordance with some applications of the present invention. As described hereinabove, for some applications, the segmented nose cone is disposed over the distal end of delivery catheter 24, in order to facilitate penetration of the interatrial septum. Subsequent to the interatrial septum having been penetrated, the segmented nose cone is retracted with respect to delivery catheter 24, such that segments 82 of the nose cone separate from each other, allowing the nose cone to be fully retracted with respect to overtube 40 of the delivery catheter, For the steps of the procedure that follow, the nose cone typically remains fully retracted with respect to overtube 40 of the delivery catheter. For some applications, the segmented nose cone is used to penetrate other portions of the subjects anatomy. For example, for procedures such as that shown in FIGS. 4A-F, the segmented nose cone may be used to facilitate penetration of the left ventricular apex.

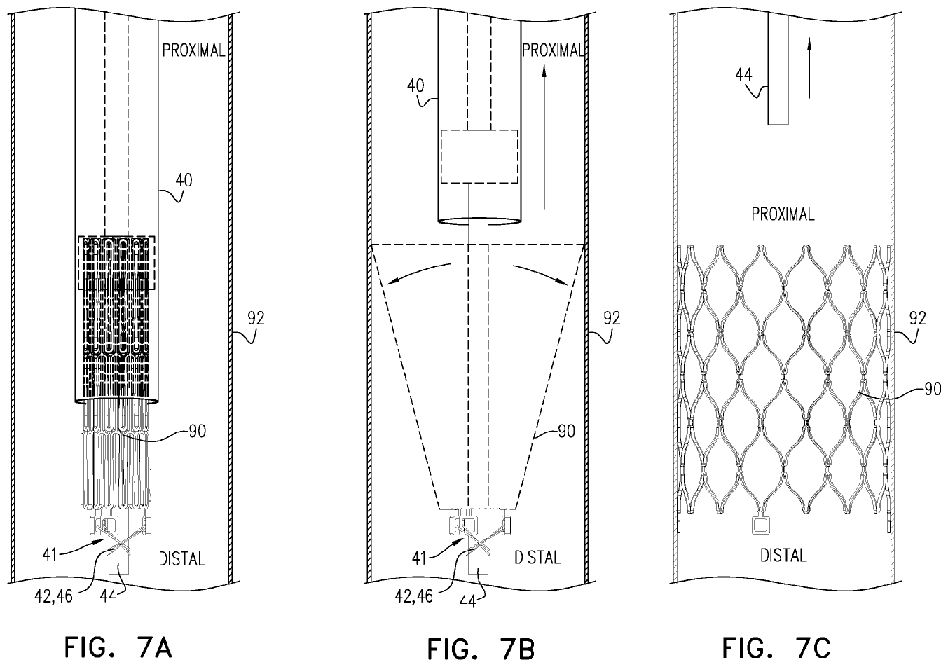

Reference is now made to FIGS. 7A, 7B, and 7C, which are schematic illustrations of respective steps of a stent 90 being deployed within a blood vessel 92, in accordance with some applications of the present invention. Typically, during delivery of the stent to the blood vessel, the proximal end of stent 90 is maintained in its radially constrained configuration by overtube 40 being disposed over the proximal end of the stent and preventing the proximal end of the stent from radially expanding, as shown in FIG. 7A. Further typically, during the delivery of the stent to the blood vessel, the distal end of the stent is maintained in its radially-constrained configuration by string-rod constraining mechanism 41. String-rod constraining mechanism 41 typically includes at least one string 42, e.g., a plurality of strings, that extend from the distal end of the stent to a rod 44 that is disposed at least partially within the overtube of the delivery catheter (e.g., a rod disposed along a longitudinal axis of the delivery catheter, as shown). For some applications, the at least one string includes a plurality of loops 46 that extend from the distal end of the stent, and that radially constrain the distal end of the stent by looping around rod 44. Typically, string-rod constraining mechanism 41 maintains the distal end of the stent in its radially-constrained configuration, even after the proximal end of the stent is allowed to assume its non-radially-constrained configuration by expanding radially (by virtue of overtube 40 being retracted from over the proximal end of the stent), as shown in FIG. 7B. For example, this may be desirable in cases in which the proximal end of the stent must be precisely positioned with respect to a portion of the patient's anatomy.

An alternative method for maintaining the distal end of the stent in its radially-constrained configuration even after the proximal end of the stent is allowed to assume its non-radially-constrained configuration would be to use a nose cone to cover the distal end of the stent. However, it would then be necessary to retract the nose cone through the stent, which may risk damaging the stent and/or dislodging the stent. Using string-rod constraining mechanism 41 to maintain the distal end of the stent in its radially-constrained configuration, rather than using a nose cone, typically reduces a risk of damaging the stent and/or dislodging the stent in this manner. This is because the rod typically has a relatively small diameter. The diameter of the rod is typically less than 9 French (3 mm), e.g., less than 6 French (2 mm). Thus, the rod can be retracted through the stent without causing any damage to or dislodging the stent.

Although some applications of the present invention are described as being utilized in conjunction with a particular type of prosthetic mitral valve and prosthetic mitral valve frame, the scope of the present invention includes using generally similar apparatus and techniques with any prosthetic mitral valve and prosthetic mitral valve frame. Similarly, although some applications of the present invention are described as being utilized in conjunction with a prosthetic mitral valve and a prosthetic mitral valve frame, the scope of the present invention includes using generally similar apparatus and techniques with any prosthetic atrioventricular valve and prosthetic atrioventricular valve. Thus, the scope of the present invention includes using generally similar apparatus and techniques with a prosthetic tricuspid valve and prosthetic tricuspid valve frame having a generally similar configuration to the prosthetic mitral valve and the prosthetic mitral valve frame described herein, mutatis mutandis. For example, a prosthetic tricuspid valve frame that includes a plurality of chord-recruiting arms may be delivered to a subject's native tricuspid valve via the subject's right atrium, using delivery catheter 24. Typically, the portion of the prosthetic tricuspid valve frame that is configured to be deployed within the subject's right ventricle is maintained in a radially-constrained configuration using a string-rod constraining mechanism 41 that includes at least one string 42 (e.g., a plurality of strings) and rod 44, in a generally similar manner to that described hereinabove. For some such applications, the at least one string comprises a plurality of loops 46 extend from the portion of the prosthetic tricuspid valve frame that is configured to be deployed within the subject's right ventricle the valve-frame body, and that loop around rod 44. The chord-recruiting arms of the prosthetic tricuspid valve frame and a portion of the prosthetic tricuspid valve frame that is configured to be deployed within the subject's right atrium are maintained in radially-constrained configurations by overtube 40 of delivery catheter 24. The chord-recruiting arms are allowed to assume non-radially-constrained configurations by retracting the overtube from over the chord-recruiting arms, while the portion of the prosthetic tricuspid valve frame that is configured to be deployed within the subject's right ventricle is maintained in its radially-constrained configuration using string-rod constraining mechanism 41. Subsequently, the portion of the prosthetic tricuspid valve frame that is configured to be deployed within the subject's right ventricle is allowed to assume its non-radially-constrained configuration by retracting rod 44, to thereby release strings 42 (e.g., loops 46).

Although some applications of the present invention are described as being utilized in conjunction with a prosthetic valve and a prosthetic valve frame, the scope of the present invention includes using generally similar apparatus and techniques with any transcatheterally-delivered medical device having a proximal portion that is configured to assume a non-radially constrained configuration while a distal portion is still to be maintained in a radially constrained configuration (e.g., stent 90, as described with reference to FIGS. 7A-C), mutatis mutandis. Typically, during the delivery of the device by the delivery catheter, the proximal portion is maintained in a radially-constrained configurations by overtube 40 of delivery catheter 24, and the distal portion of the device is maintained in a radially-constrained configuration using a string-rod constraining mechanism 41 that includes at least one string 42 (e.g., a plurality of strings) and rod 44, in a generally similar manner to that described hereinabove. For some such applications, the at least one string comprises a plurality of loops 46 that extend from the distal portion of the device, and that radially constrain the distal portion of the device by looping around rod 44. The proximal portion is allowed to assume its non-radially-constrained configuration by retracting the overtube from over the proximal portion, while the distal portion of the device is maintained in its radially-constrained configuration using the string-rod constraining mechanism. Subsequently, the distal portion of the device is allowed to assume its non-radially-constrained configuration by retracting rod 44, to thereby release strings 42 (e.g., loops 46).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a medical device; and
a delivery catheter that is configured deliver the medical device to a portion of a body of a subject, the delivery catheter comprising:
an overtube configured, during delivery of the medical device to the portion of the subject's body, to maintain a proximal portion of the medical device in a radially-constrained configuration by covering the proximal portion of the medical device; and
a string-rod constraining mechanism comprising:
a rod disposed at least partially within the overtube; and
at least one string,
the string-rod mechanism being configured to maintain a distal portion of the medical device in a radially-constrained configuration, when the overtube is not covering the distal portion of the medical device, by the at least one string extending from the distal portion of the medical device to the rod.

2. The apparatus according to claim 1, wherein the delivery catheter is configured to release the distal portion of the medical device from being maintained in the radially-constrained configuration by the rod being retracted such as to release the at least one string.

3. The apparatus according to claim 1, wherein the at least one string comprises a plurality of loops extending from the distal portion of the medical device and that are configured to maintain the distal portion of the medical device in the radially-constrained configuration by looping around the rod.

4. The apparatus according to claim 1, wherein the delivery catheter is configured to maintain the distal portion of the medical device in the radially-constrained configuration when the overtube is not covering the distal portion of the medical device, without using a nose cone to maintain the distal portion of the medical device in the radially-constrained configuration.

5. The apparatus according to claim 1, wherein the medical device comprises a stent.

6. The apparatus according to claim 1, wherein the medical device comprises a prosthetic tricuspid valve, the prosthetic tricuspid valve comprising:
a valve-frame body that defines a ventricular portion that is configured to de disposed within a right ventricle of the subject, and atrial portion that is configured to be disposed inside a right atrium of the subject;
a plurality of leaflets that are coupled to the valve frame body; and
a plurality of chord-recruiting arms that are configured to extend radially from the ventricular portion of the valve frame body.

7. The apparatus according to claim 1, wherein the medical device comprises a prosthetic mitral valve, the prosthetic mitral valve comprising:
a valve-frame body that defines a ventricular portion that is configured to de disposed within a left ventricle of the subject, and atrial portion that is configured to be disposed inside a left atrium of the subject;
a plurality of leaflets that are coupled to the valve frame body; and
a plurality of chord-recruiting arms that are configured to extend radially from the ventricular portion of the valve frame body.

8. The apparatus according to claim 7, wherein:
the prosthetic mitral valve is configured to be delivered to a native mitral valve of the subject from above the native mitral valve,
during the delivery of the prosthetic mitral valve to the subject's native mitral valve, the ventricular portion of the valve frame body is configured to be disposed distally with respect to the atrial portion of the valve-frame body, within the delivery catheter, and
the at least one string is configured to extend from a distal end of the ventricular portion of the valve-frame body to the rod.

9. The apparatus according to claim 8, wherein the at least one string comprises a plurality of loops extending from the distal end of the ventricular portion of the valve-frame body and that are configured to maintain the ventricular portion of the valve frame body in a radially-constrained configuration by looping around the rod.

10. The apparatus according to claim 7, wherein:
the prosthetic mitral valve is configured to be delivered to a native mitral valve of the subject from below the native mitral valve,
during the delivery of the prosthetic mitral valve to the subject's native mitral valve, the atrial portion of the valve frame body is configured to be disposed distally with respect to the ventricular portion of the valve-frame body, within the delivery catheter, and
the at least one string is configured to extend from a distal end of the atrial portion of the valve-frame body to the rod.

11. The apparatus according to claim 10, wherein the at least one string comprises a plurality of loops extending from the distal end of the atrial portion of the valve-frame body, and that are configured to maintain the atrial portion of the valve frame body in a radially-constrained configuration by looping around the rod.

12. A method for use with a medical device comprising:
delivering the medical device to a portion of a body of a subject using a delivery catheter, while (a) a proximal portion of the medical device is maintained in a radially-constrained configuration by an overtube of the delivery catheter covering the proximal portion of the medical device, and (b) a distal portion of the medical device is maintained in a radially-constrained configuration, by a string-rod constraining mechanism that comprises at least one string that extends from the distal portion of the medical device to a rod that is disposed at least partially within the overtube; and
when a distal end of the delivery catheter is disposed at the portion of the subject's body:
allowing the proximal portion of the medical device to assume a non-radially-constrained configuration, by retracting the overtube from over the proximal portion of the medical device, while maintaining the distal portion of the medical device in its radially-constrained configuration, by the at least one string extending from the distal portion of the medical device to the rod; and
subsequently, allowing the distal portion of the medical device to assume a non-radially-constrained configuration, by retracting the rod, to thereby release the at least one string.

13. The method according to claim 12, wherein the at least one string includes a plurality of loops extending from the distal portion of the medical device, and wherein delivering the medical device to the portion of the subject's body using the delivery catheter comprises delivering the medical device to the portion of the subject's body using the delivery catheter, while the distal portion of the medical device is maintained in the radially-constrained configuration by the plurality of loops looping around the rod.

14. The method according to claim 12, wherein delivering the medical device to the portion of the subject's body, while the distal portion of the medical device is maintained in the radially-constrained configuration comprises delivering the medical device to the portion of the subject's body, without using a nose cone to maintain the distal portion of the medical device in the radially-constrained configuration.

15. The method according to claim 12, wherein delivering the medical device to the portion of the subject's body using the delivery catheter comprises delivering a stent to a blood vessel using the delivery catheter.

16. The method according to claim 12, wherein delivering the medical device to the portion of the subject's body using the delivery catheter comprises delivering a prosthetic tricuspid valve to the portion of the subject's body using the delivery catheter, the prosthetic tricuspid valve including:
a valve-frame body that defines a ventricular portion that is configured to be disposed within a right ventricle of the subject, and atrial portion that is configured to be disposed inside a right atrium of the subject;
a plurality of leaflets that are coupled to the valve frame body; and
a plurality of chord-recruiting arms that are configured to extend radially from the ventricular portion of the valve frame body.

17. The method according to claim 12, wherein delivering the medical device to the portion of the subject's body using the delivery catheter comprises delivering a prosthetic mitral valve to the portion of the subject's body using the delivery catheter, the prosthetic mitral valve including:
a valve-frame body that defines a ventricular portion that is configured to be disposed within a left ventricle of the subject, and atrial portion that is configured to be disposed inside a left atrium of the subject;
a plurality of leaflets that are coupled to the valve frame body; and
a plurality of chord-recruiting arms that are configured to extend radially from the ventricular portion of the valve frame body.

18. The method according to claim 17, wherein delivering the prosthetic mitral valve to the portion of the subject's body using the delivery catheter comprises delivering the prosthetic mitral valve to a native mitral valve of the subject, from above the subject's native mitral valve, with the ventricular portion of the valve frame body disposed distally with respect to the atrial portion of the valve-frame body within the delivery catheter, and with the at least one string extending from a distal end of the ventricular portion of the valve-frame body to the rod.

19. The method according to claim 18, wherein the at least one string of the string-rod constraining mechanism includes a plurality of loops extending from the distal end of the ventricular portion of the valve-frame body, and wherein delivering the prosthetic mitral valve to the subject's native mitral valve comprises delivering the prosthetic mitral valve to the subject's native mitral valve, while the distal end of the ventricular portion of the valve-frame body is maintained in the radially-constrained configuration by the plurality of loops looping around the rod.

20. The method according to claim 17, wherein delivering the prosthetic mitral valve to the portion of the subject's body using the delivery catheter comprises delivering the prosthetic mitral valve to a native mitral valve of the subject, from below the subject's native mitral valve, with the atrial portion of the valve frame body disposed distally with respect to the ventricular portion of the valve-frame body within the delivery catheter, and with the at least one string extending from a distal end of the atrial portion of the valve-frame body to the rod.

21. The method according to claim 20, wherein the at least one string of the string-rod constraining mechanism includes a plurality of loops extending from the distal end of the atrial portion of the valve-frame body, and wherein delivering the prosthetic mitral valve to the subject's native mitral valve comprises delivering the prosthetic mitral valve to the subject's native mitral valve, while the distal end of the atrial portion of the valve-frame body is maintained in the radially-constrained configuration by the plurality of loops looping around the rod.
